(19) United States
(12) Patent Application Publication
(10) Pub. No.: US 2005/0287616 A1
(43) Pub. Date: Dec. 29, 2005

(54) Method of assaying denaturation of proteins

(76) Inventors: Adam F. Gross, Pleasanton, CA (US); Brendi M. Cumberland, Pleasanton, CA (US); Kelly A. Dickson, Pleasanton, CA (US); Steven E. Bromberg, Pleasanton, CA (US)

Correspondence Address: THE CLOROX COMPANY, 1221 BROADWAY PO BOX 2351, OAKLAND, CA, 94623, US

(21) Appl. No.: 10/880,783

(22) Filed: Jun. 29, 2004

(51) Int. Cl. G01N 33/53; G01N 33/542; G01N 33/537

(52) U.S. Cl. 435/7.92

(57) ABSTRACT

The present invention encompasses a method for performing an antibody-antigen assay using an ion-exchange technique prior to performing an antibody-antigen assay. Actives from protein-denaturing solutions are physically removed from the test sample, preferably by using an ion-exchange resin. The present invention also includes a method of performing an antibody-antigen assay, which does not use coating antibody on the sample holder. These methods for removing or eliminating interferences can be used in an ELISA to more effectively, efficiently and promptly screen formulations containing protein-denaturing compounds for efficacy against proteins in common allergens and detect and quantify such proteins.

### FIELD OF THE INVENTION

The present invention relates to methods for assaying the effects of protein-denaturing agents, and allows for both qualitative and quantitative measurements of the efficacy of such actives. In particular, the invention contemplates the use of antibody-antigen assays, and achieves the desired result by techniques for minimizing or eliminating interferences in antibody-antigen assays of test samples containing protein-denaturing compounds. Effective screening of efficacy of protein-denaturing compounds is achieved via these methods. The inventive methods will allow quantification of product efficacy based on contact time with allergens. Additionally, the method affords a means of studying the effects of protein-denaturing compounds on various types of proteins, especially antigens. The invention can be used in conjunction with enzyme-linked immunosorbent assays (ELISA), as well as in other immunological test methodologies such as radioimmunoassays, chemiluminescent immunoassays, fluorescent immunoassays and Radioallergosorbent tests (RAST).

### BACKGROUND OF THE INVENTION

ELISA is a fundamental tool of clinical immunology that is used as an initial screen for antigen or antibody detection. ELISAs are highly sensitive and they have become one of the most commonly used tests for detection and quantification of proteins in common allergens. The ELISA method is based on measuring the amount of binding between an antigen and an antibody specific for that antigen and employing a label enzyme and substrate to produce an amplified signal for analyte quantitation. By using a standard curve, the amount of antibody-antigen binding can be transformed in to a concentration of protein in solution. The greatest challenge in using ELISAs to screen for efficacy of protein-denaturing compounds/chemicals has been that the chemicals used to denature antigen proteins also denature other proteins, including the antibodies in an ELISA. An example of sample preparation for an ELISA consists of mixing a candidate formula and antigen, allowing them to react for a predetermined amount of time, then adding the mixture to sample container (typically a well in a 96-well microtiterplate) coated with capture antibody. Unfortunately, an unknown amount of antibody is destroyed when adding a sample containing a protein-denaturing formula to an ELISA.

Antigens are defined as those substances which trigger an antibody response, and a specific class of antigens are allergens, defined as those which trigger an allergic response. Antigens may be proteins, haptens, small molecules such as hormones, and drugs such as theophylline. All of these are considered to be within the class of antigens as defined herein. Additionally, unless otherwise clear from the context that one of these terms is intended, antigen and allergen may be used generally interchangeably herein. Antigens and allergens and their effects are more fully described in co-pending U.S. patent application Ser. No. 10/817528, filed 2 Apr. 2004, commonly owned by the assignee of the present invention, the disclosure of which is incorporated by reference herein.

Three methods have been employed to address the problem, in a prior art ELISA, of the destruction of an unknown amount of antibody. The first method involves chemically neutralizing the active in the formula to prevent the destruction of the antibody. For example, sodium sulfite can be added to an anti-allergen formula containing oxidizing agents, such as bleach or peroxide solutions, to neutralize any actives. Although chemical neutralization can be effective in the context of screening for some anti-allergen formulas, it can be ineffective in other instances, for example, when certain salts or surfactants are used in the anti-allergen formulas for which screening is desired. Surfactants are advantageous in anti-allergen formulas because they are safe for treating soft surfaces, whereas other formulas protein denaturing agents can stain or otherwise harm soft surfaces.

Dialysis is another method for the removal of active species that cause test interferences. This method is impractical for testing efficacy of an anti-allergen spray or wipe because it requires a long (as much as fifteen-hour) contact time between the antigen and the allergen denaturing formula.

A third method utilizing an interference control may be used to quantify the destruction of antibody. In this method, sample containers, e.g. microtiter plate wells are coated with capture antibody, then the test formula is added to selected wells and allowed to incubate for the same amount of time as the antigen would normally incubate in the well. Next, a known amount of antigen is added to the well and the sample is run through the rest of the ELISA. The resulting amount of antigen detected by the ELISA from the interference assay is used as a comparison point against which all of the other antigen level results are measured. Instead of removing the interfering species, the interference control only measures the maximum amount of damage the test formula causes to the assay results. The problem with the interference method is that some anti-allergen formulas destroy the entire coating antibody, which produces an unreliable control sample.

Given the problems associated with immunoassaying protein-denaturing agents, a significant need still exists for methods that allow for both qualitative and quantitative measurements of the efficacy of such agents. Quantification of the efficacy of protein-denaturing agents, based upon contact time, is not taught in the art.

There further exists a need for methods of studying the effects of protein-denaturing compounds on various types of proteins, especially antigens.

Moreover, the art is deficient in teaching an immunoassay method that allows for assaying a varying purity range of proteins, especially antigens. Thus, samples of protein, from a variety of sources, and which may be contaminated with impurities, can nonetheless be accurately assayed with the inventive method.

Similarly, in view of the prior art methods to remove or adjust ELISA results to account for interferences caused by chemicals, a significant need still exists for methods to remove interferences or minimize their impact on ELISA testing.

### SUMMARY OF THE INVENTION

The present invention provides an immunoassay method for both qualitative evaluation and quantitative measurement of the effects of protein-denaturing agents. “Protein denaturing” is defined herein to include any chemical agent, or energetic source (such as radiation) that diminishes or destroys one or more functional sites of a protein. This includes both conformational changes of the protein, as well as fragmentation thereof. The present invention includes two approaches to prevent the destruction of coating antibodies to minimize error in immunoassays resulting from use of certain chemical agents, or energetic sources. The first approach (the “ion-exchange” method) involves applying a protein-denaturing solution to a protein sample to create a test sample; physically removing any interfering chemical species from the test sample preferably by using an ion-exchange substance to create a prepared sample; adding the prepared sample into a sample container (for example, a well of a microtiter plate) coated with capture antibody; washing the sample container to remove any unbound antigen; adding a secondary labeled antibody and incubating for a predetermine amount of time; washing the sample container to remove excess labeled antibody; adding an enzyme conjugate to the prepared sample; washing the sample container to remove any unbound enzyme conjugate; adding an enzyme substrate and measuring the amount of resulting product. The resulting product concentration is calculated against a standard curve.

The second approach of the present invention (the “antibody capture” method) includes a method for reducing or eliminating the effect of interferences in an antibody-antigen assay comprising: coating microtiter plate wells with protein/antigen of interest; washing the microtiter plate wells to remove any unbound antigen; applying a denaturing solution to the protein coated wells to create a test sample; washing the microtiter plate wells to remove denaturing solution; adding labeled antibodies for a predetermined amount of time; adding an enzyme conjugate to the prepared sample; washing the microtiter plate wells to remove any unbound conjugate; adding an enzyme substrate and measuring the amount of resulting product against a standard curve.

Effective screening of efficacy of protein-denaturing compounds is achieved via these methods. Allergens, especially environmental allergens, are troublesome, and potentially harmful to a significant proportion of the population. Domestic animals are also sensitive to allergens, and in the case of commercial (farm) animals, can result in economic loss. Screening permits the development of safe and effective anti-allergen products. The inventive methods herein also afford a means of studying the effects of protein-denaturing compounds on various types of proteins, especially antigens. Either approach outlined above can be used to stop the chemical reaction between denaturing agent and protein, thus to stop denaturation of the protein. This ability to stop the reaction affords the ability to create a kinetic curve, which allows quantification of the denaturing effects over time. These methods allow quantification of product efficacy based on contact time with allergens.

### DESCRIPTION OF THE DRAWINGS

FIG. 1 is a depiction of a prior art sandwich ELISA test, employing a chromogenic label, and absent any protein-denaturing agent;

FIG. 2 is a depiction of the prior art sandwich ELISA test of FIG. 1, demonstrating the deleterious effect of a protein-denaturing agent upon the bound antibody;

FIG. 3 is a depiction of an antibody-capture method of the present invention, employing a chromogenic label, and absent any protein-denaturing agent; and

FIG. 4 is a depiction of the inventive antibody-capture method demonstrating the undisruptive effect of a protein-denaturing agent upon the bound antibody.

FIG. 5 is a depiction of protein-denaturing agent removal, showing the ion-exchange step of the method of the present invention;

FIG. 6 is a flowchart representation of a process of one embodiment of the present invention; and

FIG. 7 is a flowchart representation of a process of a second embodiment of the present invention.

### DETAILED DESCRIPTION OF THE INVENTION

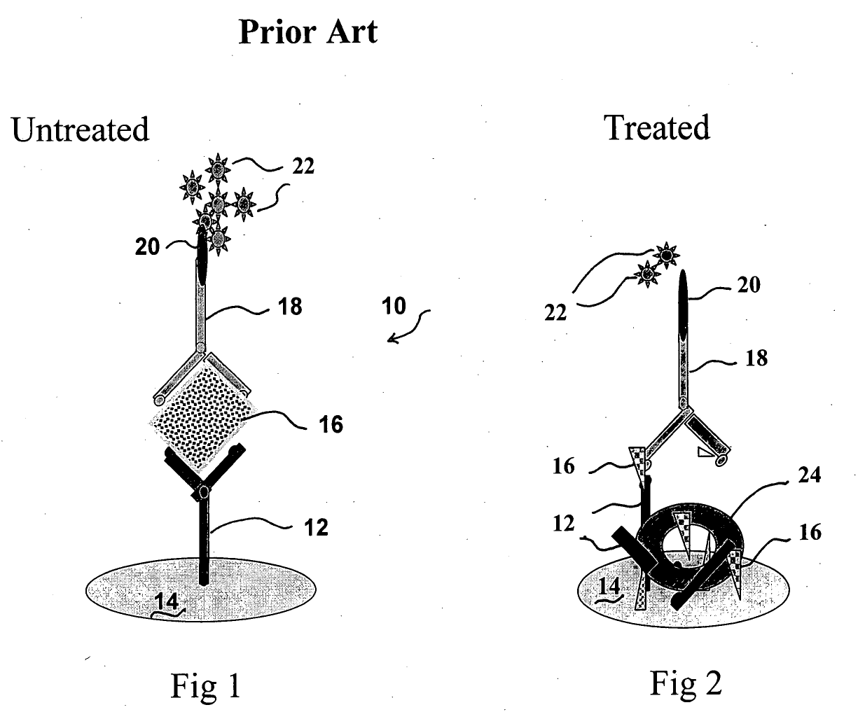

The present invention is designed to achieve accurate antibody-antigen assay test results with a test sample containing protein-denaturing agents, materials, chemicals, or compounds. The method is also suitable for assessing protein-denaturing effects of a non-chemical nature, for example, energetic sources such as radiation, electromagnetic fields, etc. A problem with both qualitative and quantitative measurements of the efficacy of protein-denaturing agents, or their effects is that they not only denature antigens, as intended, but they also denature the coating antibodies. When the protein denaturing solution destroys or inactivates coating antibodies the antibodies are no longer as effective in the ELISA test for measuring the amount of antigen in the sample. FIG. 1 illustrates a typical prior at sandwich ELISA 10, wherein an antigen-specific coating antibody 12 is bound to a substrate 14, to which the antigen 16 binds. A secondary antibody 18 binds to and “captures” the antigen 16. An enzyme conjugate 20 and label 22 complete the complex. FIG. 2 illustrates the adverse effect of an active 24 upon the coating antibody 12. Here the active 24 has denatured the antigen 16, as well as the capture antibody 12. The rate of destruction of the antibodies may differ from that of the antigen, and be either more or less rapid than that of the antigen. Because of this phenomenon, ELISA test results can show a false negative result, which makes the protein denaturing solution appear to be less effective against the targeted protein than it is in fact. A false negative is obtained when the measured reduction in the protein, for example an antigen, is less than the actual reduction thereof. Put another way, a false negative results when a very low baseline of antigen-antibody binding is detected.

In a first embodiment of the invention, the amount of test interference to a prepared sample is reduced and then the prepared sample is quantified to yield more accurate test results. The amount of actives, which cause the test interferences, must be reduced prior to the ELISA because in some cases so much of the detection antibodies would be destroyed by the protein-denaturing solution that the ELISA results would be rendered useless. This embodiment is particularly suitable for use on chemical species that form a salt. More specifically, these species include, but are not limited to, quaternary ammonium compounds and surfactants, or compounds possessing a net charge. The active species that denature proteins in anti-allergen compositions are salts and more specifically salts of quaternary ammonium compounds. Examples of suitable salts and quaternary ammonium compounds that would be appropriate for use in the present invention are discussed in detail in Kirk-Othmer Encyclopedia of Chemical Technology 3^{rd}, Vol. 22, the disclosure of which is incorporated herein by reference. The inventive methods disclosed herein permit immunoassaying protein-denaturing actives ranging from about 0.001% to about 10%, and are particularly effective in the 0.01 to 1% range, all by weight of the protein-denaturing solution. The test is thus effective over a 5 log active range, and particularly effective over a 2 log range.

The method of the present invention utilizes physical separation method using an ion-exchange technique to remove actives from an ELISA test sample. Ion-exchange techniques can be carefully chosen and used to optimize their removal actives, while still retaining the slightly negatively charged antigen in the test sample. The physical separation may be accomplished using ion-exchange substances, or resins, membranes and any other similar techniques known in the field. The ion-exchange resins may be used in a column where the test sample is run through the column prior to ELISA testing or the resin may be placed directly into the test sample holders prior to ELISA testing.

The ion-exchange resin used in the present invention may be cationic, anionic or amphoteric. Different resins must be selected to remove different actives from the test sample. In one embodiment, the resins are organic and polymeric, such as those sold by the Dow Chemical Company under the trademark Dowex, and in another embodiment, the resins are inorganic such as aluminosilicate zeolites. Silica gel resins are one such example. The type of resin used for the ion-exchange will determine how much of the resin is needed to remove the actives from the test sample. For each ion-exchange resin, the manufacturer will list the prescribed resin capacity. The prescribed resin capacity is the total number of chemical equivalents available for exchange per some unit weight or unit volume of resin. The capacity may be expressed in terms of milliequivalents per dry gram of resin or in terms of millequivalents per milliliter of wet resin. In a preferred embodiment of the invention, the level of actives in the test solution is less than 90% of the prescribed resin capacity.

The actives in the test sample are separated out using an ion-exchange resin, resulting in a prepared sample. The prepared sample is then placed in a sample holder. The sample holder may be a tube or microtiter plate wells to allow for parallel processing of many samples or any other sample holder known in the field. In one embodiment of the invention, the sample holders are coated with antibody specific to the antigen being tested. When the sample is applied to the holder the coating antibody reacts with the antigen binding the two together so that when the sample holder is subsequently washed the antibody-antigen complex is retained in the sample holder. Next, an enzyme conjugate specific for the antigen is added to the prepared sample and the prepared sample is washed once again. Finally, the amount of resulting antibody-antigen complex is measured by adding an enzyme substrate, which indicates the amount of resulting product by color using a chromophore or by other radioactive or fluorescent labeling techniques. The fluorescent or radioactive labeling may include a technique for self-amplification.

In a second embodiment of the invention, an antibody-antigen assay is performed without utilizing capture antibodies on the test sample vessel. Since no capture antibody is utilized, test interferences are eliminated, and it is therefore unnecessary to inactivate or adjust protein-denaturing solutions or to remove the actives from the test sample prior to an ELISA. In the method of this embodiment, the antigen is first bound directly onto a solid surface, such as that of a microtiter plate well, or other suitable sample holder. Next, the protein-denaturing solution is added and allowed to react with the antigen for a set time. The sample holder is then washed to remove any residual product containing chemical actives. Next, labeled, antigen specific antibodies are added to the wells or sample vessel and allowed to form a complex. An enzyme conjugate is added to the prepared sample and the prepared sample is washed once again to remove excess reagent. Finally, the amount of resulting antibody-antigen complex is measured by adding an enzyme substrate, which indicates the amount of resulting product by means of colorimetry. This method is also applicable when using fluorescent or radioactive labels. This method is effective with all classes of antibodies, and is effective when using antibody capture ELISAs where the antibody is of the IgE class.

Since this embodiment of the invention eliminates the use of capture antibodies, it takes significantly less time than conventional and interference ELISAs, thus making the screening of protein denaturing substances in solution a more rapid process. Not only does the antibody-antigen assay method of the present invention give more accurate and direct results but it is also significantly more efficient than the conventional and interference ELISA methods.

Both the first and second embodiments of the present invention are not limited to ELISA testing for anti-allergen compounds. The present invention relates to any methods for minimizing or eliminating interferences in any antibody-antigen assay for samples with protein-denaturing compounds. Although the primary focus of this invention is on improved methods for enzyme-linked immunosorbent assays (ELISA) these improved methods can also be used in radioimmunoassays, fluorescent immunoassays and Radioallergosorbent tests (RAST) or other suitable tests known in the art, such as colorimetric tests.

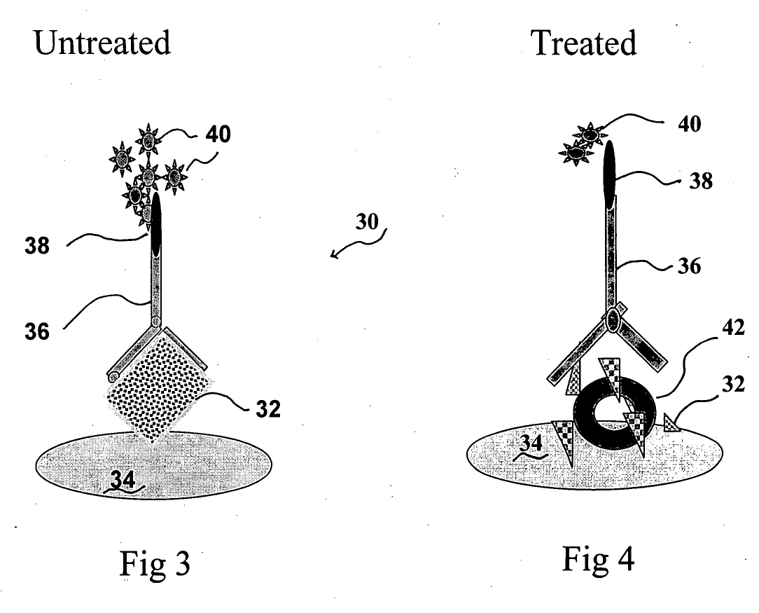

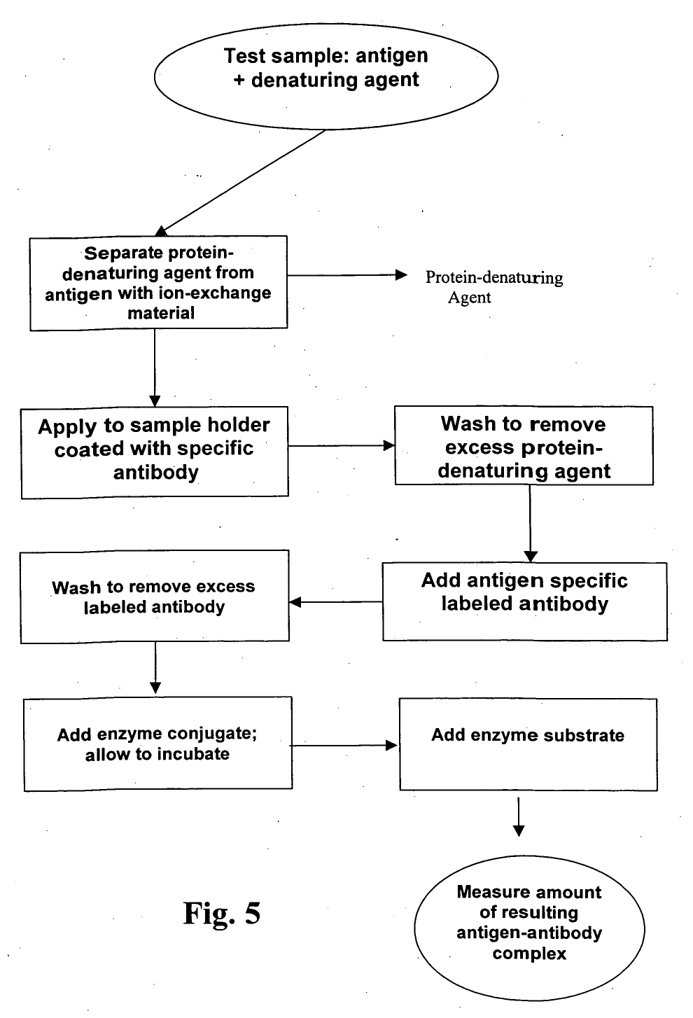

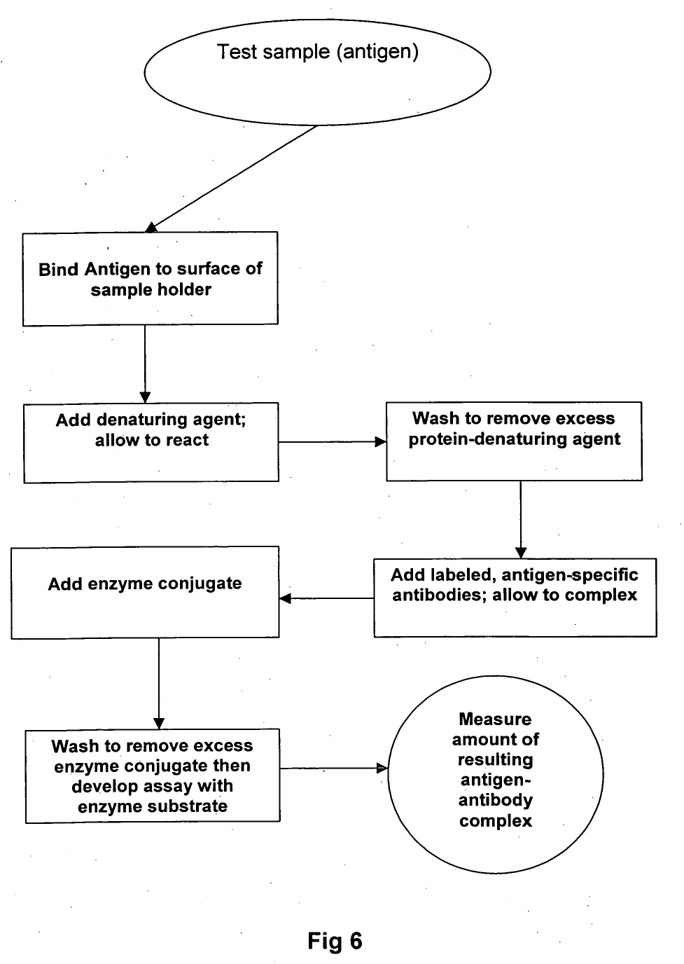

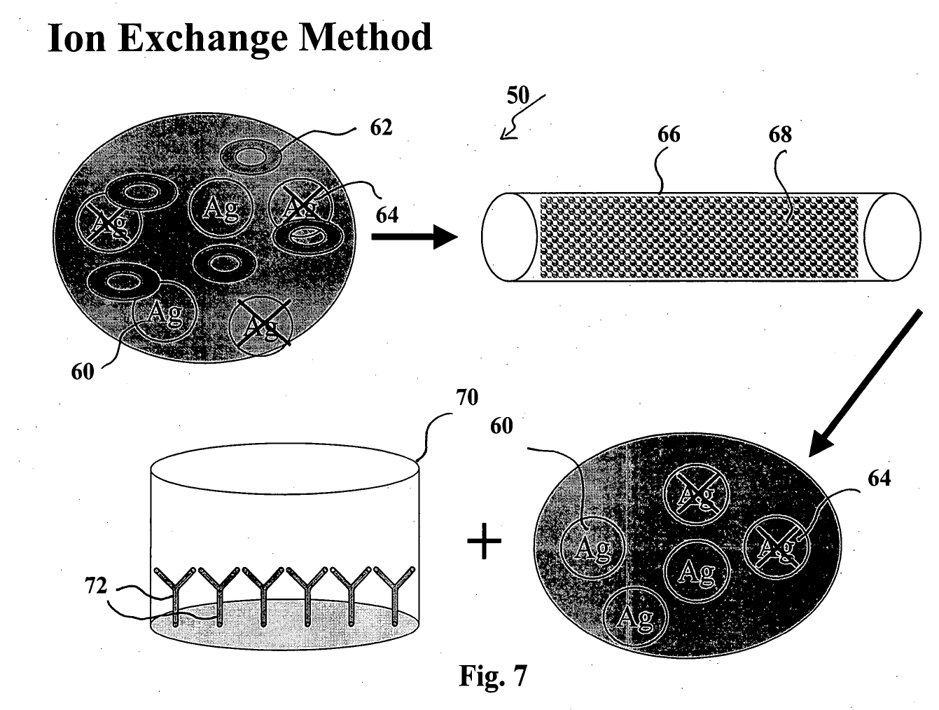

FIGS. 3 and 4 illustrate an antibody-antigen assay 30 of the present invention, wherein the antibody capture method is employed. In FIGS. 3 and 4, a coating antibody is not used; rather an antigen 32 is bound to a substrate 34. An antigen-specific antibody 36 binds to the antigen 32, and an enzyme conjugate 38 and label 40 bind to the antibody 36 and enzyme conjugate 38, respectively. Because the inventive method requires no coating antibody bound to the substrate 34, there can be no destruction of coating antibody, therefore no inherent errors in the process. FIG. 4 graphically illustrates the limitation of the effect of an active 42 to the target antigen 32 only. FIG. 5 is a depiction of the portion of the ion-exchange method of the present invention wherein an ion-exchange resin is used to physically remove a protein-denaturing agent from a mixture containing test antigen. In the Fig., the process 50 includes the step of contacting antigen 60 with a protein-denaturing agent 62. This results in a mixture of some active antigen 60, denaturing agent 62, and some denatured or inactivated antigen 64. The mixture is then passed through an ion-exchange column 66, containing suitable ion-exchange resin 68 as known to the art. Eluting from the column 66 is a mixture of active antigen 60, and denatured or inactivated antigen 64, which is then incubated in a sample holder 70 containing a plurality of coating antibody 72. The assay is concluded as described herein.

It should be noted that ion-exchange resins can be selected to either bind protein (antigen) and elute protein-denaturing agent, or can bind protein-denaturing agent and elute protein. In particular, with the latter, some ion exchange resins will bind both protein and denaturing agent, but protein can be freed from the resin (retaining denaturing agent) by eluting with a buffered solution. Since a protein's net charge is influenced by pH, changing the pH results in a change in charge, allowing the protein to elute. An uncharged denaturing agent will exhibit pH independent interactions with the resin.

Both the ion exchange and antibody capture methods allow control of reaction time. For the ion-exchange method, it is possible to use ion-exchange columns that will extract all, or substantially all, of the protein-denaturing active in less than 30 seconds, or in less than 20 seconds, or in less than 10 seconds, or in less than 5 seconds. Other ion-exchange columns will extract all, or substantially all, of the protein-denaturing active in less than 3 hours, or in less than 2 hours, or in less than 1 hour. Once the antigen/active solution is drawn through the ion-exchange column, all active will be removed and the reaction will be stopped. This step can be done any time after mixing the antigen and active. For the antibody capture method, washing the plate at any time after the active is added stops the reaction. Both methods allow short contact times between active and antigen, which was previously not possible for salt and surfactant based actives.

Control of reaction times affords a variety of benefits. When screening protein-denaturing agents for applications in surface cleaning products, the experimenter can mimic drying times' actual use, for example, in consumers' homes. Additionally, kinetic curves can be created that show both the effect of concentration on denaturation, and also the effect of time. This permits determining a minimum reaction and a minimum drying time required to inactivate a particular allergen/antigen, which relates to product efficacy. Furthermore, kinetic curves can be fitted to kinetic rate laws, which allow mathematical predictions of the effect of concentration and elapsed time on efficacy.

### Experimental Results

To assess the efficacy of the methods of the present invention, a series of experiments were first done to quantify the interference caused by protein-denaturing compounds in a prior art ELISA. Since the highly destructive actives that cause the interference can also lead to false negative results, test samples were run through a column of ion-exchange resin to reduce the amount of actives prior to performing the ELISA tests. Since the ion-exchange resin also retains some of the allergen protein of interest, tests were performed to determine the amount of allergen lost from the test samples. Using only the necessary amount of resin can optimize the process because the less resin used in the ion-exchange process the less allergen protein of interest that is retained in the resin, thus leading to great improvements in the accuracy of the tests.

Table I below shows the effects of various surfactant-based protein-denaturing actives on a Der p1 allergen, utilizing the ion-exchange resins method of the present invention.

TABLE 1
Effect of Ion-Exchange Resin in Removal of Actives

| | Allergen Level in | Allergen Level in | |
|---|---|---|---|
| | Active Solution | Active Solution with | |
| | Passed Through | Der p1 Then Passed | Percent |
| | Resin Then Mixed | Through Resin | Reduction of |
| Active | with Der p1 (ng/ml) | (ng/ml) | Allergen |
| Bardac 2050 | 138 | 5 | 93% |
| Lonzabac 12 | 161 | 26 | 69% |
| Bardac 2050 + | 120 | 5 | 92% |
| Lonzabac 12 | | | |

Once the effect of the ion-exchange resin on the allergen protein was determined, tests were performed to determine the amount of reduction in actives by the ion-exchange resin. Samples of active solutions diluted with water were prepared using 0.4% Bardac 2050 by weight, 0.15% Lonzabac 12 by weight, and 0.4% Bardac 2050 plus 0.15% Lonzabac 12 by weight. These chemicals are all sold by Lonza, Inc., Fairlawn, N.J. The prepared active solutions were run through a column of Dowex MR-3 resin. For one set of active test samples, Der p1 allergen (a commercially-available recombinant protein of the Indoor Biotechnologies company) was added after passing the solution through the ion-exchange resin. For the second set of active test samples, Der p1 allergen was added prior to passing the samples through the ion-exchange resin. Fourier Transform Infrared Spectroscopy (FTIR) showed that less than 1% of the original Bardac 2050 and less than 7% of the original Lonzabac 12 remained after passing the active test samples through the ion- exchange resin. Using the inventive method of minimizing the interference effect of the actives, and by taking into account the allergen retained in the resin, the percentage reduction in allergen caused by the actives could be accurately measured, as shown in Table I. Allergens were measured by optical density at a single wavelength, and concentrations were determined by calculating against a standard curve using a Molecular Devices SOFTmax PRO (version 3.1), and a percentage reduction was generated by Equation 1:

$$\frac{[ control - test sample ]}{[ control ]} \times 100 = \% reduction \qquad ( 1 )$$

Table II below demonstrates the accuracy improvement in measuring actual allergen inactivation (on a Der p1 allergen) using the ion- exchange method of the present invention compared with the prior art interference method. In other respects, the experimental methods were as described for Table I.

TABLE II
Comparison of Methods

| | Percent Allergen | |
|---|---|---|
| | Reduction Measured By | Percent Allergen Reduction |
| | Prior Art Interference | Measured By Ion- |
| Active | Control | Exchange Method |
| Bardac 2050 | 47% | 93% |
| Lonzabac 12 | 0% | 69% |
| Bardac 2050 + | 74% | 92% |
| Lonzabac 12 | | |

For comparison purposes, ELISAs were run with interference controls and with an ion-exchange step prior to the ELISA. The interference control was prepared by allowing the active test solution to be added to the sample holder for the same amount of time that the antigen sample and then a known amount of antigen is added and an ELISA is performed. The following is a more detailed description of a specific interference control ELISA of the prior art:

Capture monoclonal antibodies are bound to polystyrene surface, and allowed to remain overnight. Excess capture antibodies are washed with a wash buffer using a microtiter plate washer. The protein-denaturing agent and capture antibodies are incubated for 1 hour prior to ELISA testing. This is used to evaluate the damage caused to the antibodies by the agent. The protein-denaturing agent is then washed off using a microtiter plate washer.

Standards and controls are prepared. Protein-denaturing agent test samples are prepared by incubating with antigen for a pre-determined period of time. An antigen solution of the same concentration as that used with the product is prepared so that it is added to the interference control wells to assist in evaluating damage to the antibodies.

Standards, Interference control antigen solution, antigen controls, product blanks and products are incubated in parallel for one hour. Excess antigen from all wells is washed off using a microtiter plate washer. A detection antibody, specific to the antigen tested, is incubated for one hour at room temperature, and the excess detection antibody is washed off. An enzyme conjugate is added and incubated at room temperature for a specified time, then excess conjugate is washed off.

A substrate such as ABTS (2,2′-AZINO-bis (3-Ethylbenzthiazoline-6-sulfonic acid) or TMB (3,3′,5,5′-Tetramethylbenzidine) is incubated for a specified period of time to develop the assay. Finally, the ELISA is read using a microtiter plate reader such as the Spectramax 250 by Molecular Devices.

The comparison of methods in Table 2 shows higher percentages for the ion-exchange method indicating that it is more effective than the interference control method at accurately measuring allergen inactivation. The effectiveness of the ion-exchange method is particularly apparent in the case of the Lonzabac 12 sample, because the interference control gave a false negative result. This false negative result happens when the actives destroy all of the coating antibody.

In contrast to the interference control method, the ion-exchange method was able to measure the effects of Lonzabac 12 on the allergen. Therefore, the inventive method demonstrates an improvement in accuracy over previously available tests.

Table III below is a comparison of three different commercially-available protein-denaturing agents on the Der p1 allergen, measured by the antibody capture test of the present invention. The antibody capture test was conducted as follows: microtiter plate wells were coated with the Der p1 antigen. The microtiter plate wells were washed to remove any unbound antigen, and a denaturing solution was applied to the protein coated wells to create a test sample. The microtiter plate wells containing test sample were washed to remove denaturing solution, labeled antibodies were added for a predetermined amount of time then an enzyme conjugate was added to the prepared sample. Next, the microtiter plate wells were washed to remove any unbound conjugate, an enzyme substrate was added and the amount of resulting product was measured against a standard curve, as described in connection with Table I.

More detail regarding the effects of the dilute (85 ppm available chloride) and the ultra dilute (8.5 ppm available chloride) hypochlorite solution used in the test of table III may be found in copending U.S. patent applications Ser. No. 10/806522, filed 23 Mar. 2004, and Ser. No. 10/828571, filed 20 Apr. 2004, both of which are commonly owned by the assignee of the present invention, the disclosures of which are incorporated by reference herein.

TABLE III
Effect of protein-denaturing actives on allergen protein

| | Protein | | | | |
|---|---|---|---|---|---|
| | Concentration | | Protein | | |
| | applied in | | concentration After | | % Reduction |
| Sample | ELISA wells | | active treatment | | of Allergen |
| DDI Water + | 2.8 | ug/mL | 2.8 | ug/mL | 0% |
| Surfactant and | | | | | |
| fragrance | | | | | |
| Dilute NaOCl | 2.8 | ug/mL | 0.016 | ug/mL | 99% |
| Bleach Solution | | | | | |
| Ultra Dilute | 7.2 | ug/mL | 3.8 | ug/mL | 47% |
| NaOCl Bleach | | | | | |
| solution | | | | | |

Table IV below is a comparison of assay time between the prior art neutralization and interference methods, and the antibody capture method of the present invention. The neutralization method employed thiosulfate as a neutralizing agent. The prior art interference method was conducted as described for Table II above. The antibody capture method of the present invention was as described in reference to Table III above. Table IV illustrates the assay time savings of the methods of the present invention.

TABLE IV
Comparison of product Efficacy Screening

| Prior Art | Prior Art | |
|---|---|---|
| Neutralization | Interference | Antibody Capture |
| ELISA | ELISA | ELISA |
| % Allergen | % Allergen | % Allergen |
| Reduction | Reduction | Reduction |
| >99% | >99% | >99% |
| 3-4 hours assay | 5-6 hours assay | 2 hours assay |
| time⁽¹⁾ | time⁽¹⁾ | time⁽¹⁾ |

⁽¹⁾Time of assay excludes overnight coating of antigen or antibody.

The testing methods described are not intended to limit in any manner the scope or equivalents to which the invention is entitled, the invention is further characterized by the claims, which follow.

1. A method of performing a protein assay in steps comprising:
(a) denaturing a protein sample to create a test sample;
(b) removing any interfering species from the test sample to create a prepared sample;
(c) adding an enzyme conjugate specific for the protein to the prepared sample, wherein a protein-enzyme conjugate complex is formed; and
(d) measuring the amount of resulting protein-enzyme conjugate complex

2. The method of assay according to claim 1, wherein the protein is an antigen.

3. The method of assay according to claim 2 wherein the protein specificity of step (c) is obtained through the use of an antibody.

4. The method of assay according to claim 3, wherein the interfering species is a chemical agent; and the step of removing comprises a physical removal.

5. The method of assay according to claim 1 wherein the protein sample is pure.

6. The method of assay according to claim 1 wherein the protein sample is impure.

7. A method of performing an antibody-protein assay in steps comprising:
(a) denaturing a protein sample to create a test sample;
(b) removing any protein-denaturing agents from the test sample to create a prepared sample;
(c) adding the prepared sample to a sample holder;
(d) washing the sample holder to remove any unbound antigen;
(e) adding an enzyme conjugate specific for the antigen to the prepared sample, wherein an antibody-enzyme complex is formed;
(f) removing any unbound enzyme; and
(g) measuring the amount of antibody-enzyme complex.

8. The method of performing an antibody-protein assay according to claim 7, wherein the protein is an antigen.

9. The method of performing an antibody-protein assay according to claim 7, wherein protein is denatured by the step of applying a denaturing agent.

10. The method of performing an antibody-antigen assay according to claim 9, wherein the protein-denaturing agent is a chemical.

11. The method of performing an antibody-antigen assay according to claim 1, wherein the sample holder is a multiple well plate allowing multiple prepared samples to be tested at the same time.

12. The method of performing an antibody-antigen assay according to claim 11, wherein the sample holder is coated with capture antibody.

13. The method of performing an antibody-antigen assay according to claim 7, wherein the step of physically removing said interfering chemical species is performed using an ion-exchange resin.

14. The method of performing an antibody-antigen assay according to claim 13, wherein interfering chemical species are physically removed from the prepared sample by the step of passing the sample through an ion-exchange resin column.

15. The method of performing an antibody-antigen assay according to claim 13, wherein interfering chemical species are physically removed from the prepared sample by the step of adding an ion-exchange resin directly into the prepared sample in the sample holder.

16. The method of performing an antibody-antigen assay according to claim 13, wherein the ion-exchange resin is a cationic, anionic or amphoteric resin.

17. The method of performing an antibody-antigen assay according to claim 13, wherein the test sample is allowed to react with the ion-exchange substance for between 5 seconds and 3 hours.

18. The method of performing an antibody-antigen assay according to claim 13, wherein the ion-exchange resin is either a polymer-based resin or a silica-containing material.

19. The method of performing an antibody-antigen assay according to claim 13, wherein the ion-exchange resin is capable of removing protein-denaturing agents generated from salts in the test sample.

20. The method of performing an antibody-antigen assay according to claim 13, wherein the level of said actives is less than 90% of the prescribed resin capacity.

21. The method of performing an antibody-antigen assay according to claim 13, wherein color, radioactive or fluorescent labeling techniques are used to measure the amount of resulting product.

22. The method of performing an antibody-antigen assay according to claim 21, wherein the labeling techniques include self-amplification.

23. The method of performing an antibody-antigen assay according to claim 10, wherein the denaturing agent comprises quaternary ammonium compounds.

24. The method of performing an antibody-antigen assay according to claim 23, wherein the ion-exchange resin is capable of removing actives generated from quaternary ammonium compounds.

25. The method of performing an antibody-antigen assay according to claim 24, wherein the quaternary ammonium compounds are about 0.001%-10% by weight of the test sample.

26. A method of performing an antibody-antigen assay in steps comprising:
(a) applying a denaturing solution to a bound protein sample to create a test sample;
(b) removing remaining denaturing solution from solution;
(c) adding detection antibodies to form an protein-antibody complex;
(d) removing unbound detection antibodies;
(e) adding an enzyme conjugate to create a prepared sample;
(f) removing any unbound enzyme;
(g) adding an enzyme substrate to generate a labeled product; and
(h) measuring the amount of resulting product.

27. The method of performing an antibody-antigen assay according to claim 26, wherein the steps of removing are achieved by washing.

28. The method performing an antibody-antigen assay according to claim 26, wherein color, radioactive, or fluorescent labeling techniques are used to measure the amount of resulting product.

29. The method of performing an antibody-antigen assay according to claim 28, wherein the labeling techniques include self-amplification.

30. The method of performing an antibody-antigen assay according to claim 26, wherein the denaturing solution comprises quaternary ammonium compounds, acids, bases, salts, oxidizers and reductants.

31. The method of eliminating the effect of interferences according to claim 26, wherein the quaternary ammonium compounds are about 0.001%-10% by weight of the test sample.
